(19) United States
(12) Patent Application Publication
(10) Pub. No.: US 2018/0147359 A1
(43) Pub. Date: May 31, 2018

(54) FILTER STRUCTURE FOR SYRINGE AND SYRINGE INCLUDING SAME

(71) Applicant: SANG-A FRONTEC CO.,LTD., Incheon (KR)

(72) Inventors: Won Hyun SEOK, Incheon (KR); Soo Min CHAE, Incheon (KR); Jung Sik MIN, Incheon (KR)

(73) Assignee: SANG-A FRONTEC CO.,LTD., Incheon (KR)

(21) Appl. No.: 15/575,805

(22) PCT Filed: Nov. 6, 2015
(86) PCT No.: PCT/KR2015/011883

§ 371 Date: Nov. 20, 2017

(30) Foreign Application Priority Data
May 20, 2015 (KR) 10-2015-0070670

(51) Int. Cl. A61M 5/31; A61M 5/165

(52) U.S. Cl. CPC A61M 5/3145; A61M 5/321; A61M 5/165

(57) ABSTRACT

A filter structure for a syringe comprises a filter unit installed within a cylinder and connected to a needle coupled to the front of the cylinder-in order to perform filtering when an injection solution introduced into the cylinder through the rear thereof is discharged through the needle, wherein a second fluid passage is formed outside a first fluid passage, and the filter unit comprises: a first check valve that is connected with the needle and blocks the second fluid passage; a second check valve that blocks the first fluid passage; a filter member installed on the second fluid passage; and a filter body located between the first check valve and the filter member and coupled to the first check valve at the front thereof and to the second check valve at the rear thereof.

### CROSS-REFERENCE TO RELATED APPLICATIONS

This application is the national phase entry of International Application No. PCT/KR2015/011883, filed on Nov. 6, 2015, which is based upon and claims priority to Korean Patent Application No. 10-2015-0070670, filed on May 20, 2015, the entire contents of which are incorporated herein by reference.

### TECHNICAL FIELD

The present invention relates to a filter structure for a syringe and a syringe including the same.

### BACKGROUND

Generally, a syringe is used for taking blood from a blood vessel of a patient or taking another body fluid from a body in addition to blood. On the other hand, a syringe is used for directly injecting a drug into a blood vessel or injecting a drug into tissue or injecting a drug hypodermically.

That is, a syringe may be used for taking a body fluid from a body or for injecting a drug into a body.

Meanwhile, when an injectable solution is injected into a body using a syringe, a top end of a glass ample or a small glass bottle with a rubber stopper, which is filled with the injectable solution, is cut and an injection needle is put through a cut part to suction the injectable solution in order to move the injectable solution to the syringe.

When an injectable solution is injected into a body using a conventional syringe, a slight amount of micro glass particles or pieces may be suctioned with the injectable solution into the syringe in such a way that both the micro glass particles and the injectable solution may be injected into the body.

As described above, when an injectable solution is injected into a patient, micro glass particles may be injected with the injectable solution into a human body, which has a bad effect on the health of the patient.

Also, in the case of a conventional syringe, when an injectable solution is suctioned, glass particles of an ample are filtered out using a filter. However, since it is necessary to remove the filter on which glass particles of the ample are present before giving an injection, an injection process is complicated.

### SUMMARY OF INVENTION

### Technical Problem

One aspect of the present invention provides a filter structure for a syringe, which simplifies an injection process and prevents glass particles of an ample from being injected into a patient by allowing an injectable solution not to pass through a filter member when being injected into a syringe and to pass through the filter member when being injected into the patient, and a syringe including the same.

### Technical Solution

According to one aspect of the present invention, there is provided a filter structure for a syringe for filtering an injectable solution that flows backward from an injection needle coupled with a front of a cylinder. The filter structure includes a filter portion in which a second fluid path through which the injectable solution discharges from the cylinder is formed on an outer circumferential portion of a first fluid path, which is installed in the cylinder, connected to the injection needle, and through which the injectable solution flows, not to allow the first fluid path to overlap with the second fluid path. Here, the filter portion includes a first check valve that is connected to the injection needle and allows the injectable solution to flow into the cylinder through the first fluid path by obstructing the second fluid path, a second check valve that obstructs the first fluid path to discharge the injectable solution outward from the cylinder through the second fluid path, a filter member that is installed at the second fluid path and filters the injectable solution that passes through the second check valve and discharges, and a filter body that is positioned between the first check valve and the filter member and has a front part coupled with the first check valve and a rear part coupled with the second check valve to allow the filter member to come into close contact with the second check valve.

Here, the filter structure may include a filter holder that is installed in the cylinder and has a front part coupled with the injection needle and a rear part with an accommodation portion in which the filter portion is accommodated. Here, the filter holder may have a cylindrical shape with a center at which a through hole through which the injectable solution passes is formed, and the through hole may be connected to the first fluid path and the second fluid path.

Here, a holding lip formed to protrude toward an inside of the filter holder may be provided at a front part of the accommodation portion to prevent the filter portion accommodated in the accommodation portion from deviating forward.

Here, an O-ring groove formed to be depressed inward for sealing a gap between an inside of the cylinder and an outer circumferential surface of the filter holder may be provided on the outer circumferential surface of the filter holder.

Here, the filter body may include a first outer barrel portion having a barrel shape, a first inner barrel portion connected to the first outer barrel portion and formed in the first outer barrel portion, and an extending pipe formed to extend backward from the first inner barrel portion.

Here, the filter body may include a first suction hole formed at the first fluid path in centers of the first inner barrel portion and the extending pipe and a first discharge hole formed at the second fluid path between the first outer barrel portion and the first inner barrel portion.

Here, a plurality of protrusions may be formed on an inner circumferential surface of the filter holder, and a plurality of insertion-grooves formed corresponding to the plurality of protrusions may be formed on an outer circumferential surface of the first outer barrel portion in such a way that the filter body may be coupled with the accommodation portion of the filter holder.

Here, the first check valve may include a discus portion that has a discus shape and is disposed to come into close contact with a front surface of the filter body to selectively obstruct the first discharge hole when the injectable solution flows in or discharges and a first column portion formed to protrude backward from the discus portion and inserted into and coupled with the first suction hole. Here, a second suction hole may be formed at the first fluid path in centers of the discus portion and the first column portion.

Here, the discus portion may be disposed to be spaced backward apart from the holding lip in such a way that a mobile space may be formed between the holding lip and the discus portion to allow the discus portion to be spaced apart from the first discharge hole.

Here, the second check valve may include a second outer barrel portion having a barrel shape and a second inner barrel portion connected to the second outer barrel portion and formed in the second outer barrel portion. Here, a third suction hole into which the extending pipe is inserted and which allows the filter member to come into close contact with a front surface of the second check valve may be formed at the first fluid path in a center of the second inner barrel portion, and a second discharge hole may be formed at the second fluid path between the second outer barrel portion and the second inner barrel portion.

Here, the filter structure may include a section including a cutting line and at least partially cut at one side of the third suction hole. Here, the section may selectively obstruct the third suction hole when the injectable solution flows in or discharges.

Here, the first discharge hole and the second discharge hole may be formed to extend in a circumferential direction and to surround at least parts of the first suction hole and the third suction hole.

According to another aspect of the present invention, there is provided a syringe including a fixing member having a front on which an injection needle is mounted, a cylinder with which the fixing member is detachably coupled, the above-described filter structure which is installed in the cylinder and coupled with the fixing member, and a piston that moves forward or backward while being at least partially inserted in the cylinder to allow an injectable solution to flow into the cylinder or to discharge outward from the cylinder.

Here, a screw thread may be formed on an inner circumferential surface of a front of the filter holder and screw-coupled with a rear part of the fixing member in such a way that at least part of the fixing member may be installed in the filter holder.

Here, the piston may include a piston body that is inserted into the cylinder and moves in one direction, a gasket coupled with a front end of the piston body and inserted into the cylinder to come into close contact therewith, and a coupling shaft member that is positioned at a front end portion of the piston body, is formed to protrude forward, and couples the gasket with the piston body.

Here, a holding protrusion with a coupling groove formed to be depressed backward may be formed at an end of the coupling shaft member. Here, a supporting lip formed to extend inward may be provided on an inner circumferential surface of the first inner barrel portion, and a rear end surface of the holding protrusion may be supported by the supporting lip when the coupling shaft member passes through the third suction hole.

Here, the holding protrusion may be formed of an elastically deformable material.

Here, the piston body may include a bent groove formed to be depressed inward between the coupling shaft member and a rear end of the piston body.

### ADVANTAGEOUS EFFECTS

According to one embodiment of the present invention, a syringe including a filter structure for a syringe may simplify an injection process and prevent foreign substances from being injected into a patient by allowing an injectable solution not to pass through a filter member when being injected into the syringe and to pass through the filter member when being injected into the patient.

According to one embodiment of the present invention, since the syringe including the filter structure prevents an injection needle from being reused and simultaneously deviating outward, a negligent accident of being stuck with the injection needle may be prevented during a process of discarding the syringe.

### BRIEF DESCRIPTION OF DRAWINGS

FIG. 1 is a perspective view of a syringe including a filter structure for a syringe, according to one embodiment of the present invention.

FIG. 2 is an exploded perspective view of the syringe including the filter structure for a syringe, according to one embodiment of the present invention.

FIG. 3 is a cross-sectional view of the syringe including the filter structure for a syringe, according to one embodiment of the present invention.

FIG. 4 is a perspective view of the filter structure for a syringe, according to one embodiment of the present invention.

FIG. 5 is a perspective view illustrating a cross-section of a filter holder of the filter structure for a syringe, according to one embodiment of the present invention.

FIG. 6 is a perspective view illustrating a cross-section of a filter body of the filter structure for a syringe, according to one embodiment of the present invention.

FIG. 7 is a perspective view illustrating a cross-section of a second check valve of the filter structure for a syringe, according to one embodiment of the present invention.

FIG. 8 is a cross-sectional view illustrating an injection of an injectable solution into the syringe including the filter structure for a syringe, according to one embodiment of the present invention.

FIG. 9 is a cross-sectional view illustrating a discharge of an injectable solution from the syringe including the filter structure for a syringe, according to one embodiment of the present invention.

FIG. 10 is a cross-sectional view illustrating a state in which a holding protrusion of the syringe including the filter structure for a syringe is coupled with a supporting lip, according to one embodiment of the present invention.

FIG. 11 is a cross-sectional view illustrating that a bent groove of the syringe including the filter structure for a syringe, according to one embodiment of the present invention is exposed outward.

### DETAILED DESCRIPTION OF INVENTION

Hereinafter, embodiments of the present invention will be described in detail with reference to the attached drawings to allow one of ordinary skill in the art to easily implement. The present invention may be embodied in several various forms and is not limited to the embodiments described herein. In the drawings, to definitely describe the present invention, parts irrelevant to the description are omitted. Throughout the specification, like reference numerals refer to like elements.

Throughout the specification, it should be understood that the terms “comprise”, “have”, etc. are used herein to specify the presence of stated features, numbers, steps, operations, elements, components or combinations thereof but do not preclude the presence or addition of one or more other features, numbers, steps, operations, elements, components, or combinations thereof. Also, when it is stated that a part of a layer, film, area, plate and the like is “on” another part, the part includes not only “being directly on” the other part but also being thereon with still another part therebetween. On the other hand, when it is stated that a part of a layer, film, area, plate and the like is “below” another part, the part includes not only “being just below” the other part but also being therebelow with still another part therebetween.

Hereinafter, a filter structure for a syringe and a syringe including the same according to one embodiment of the present invention will be described in detail. Also, in one embodiment, an injectable solution refers to a material including a mixture of a medicinal fluid, glass particles and the like.

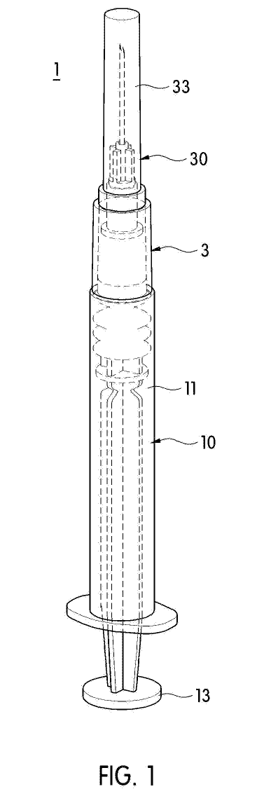

FIG. 1 is a perspective view of a syringe including a filter structure for a syringe, according to one embodiment of the present invention.

In a following description, it will be described that a direction from a piston to a cover is defined as front and a direction from the cover to the piston is defined as rear in FIG. 1.

Referring to FIG. 1, a syringe 1 including a filter structure 3 for a syringe according to one embodiment of the present invention may include an injection needle hub 30, a cylinder 11, a piston 10, and a filter structure 3.

According to one embodiment of the present invention, the syringe 1 including the filter structure 3 for a syringe may simplify an injection process and prevent foreign substances from being injected into a patient by allowing an injectable solution not to pass through a filter member when being injected into the syringe and to pass through the filter member when being injected into the patient.

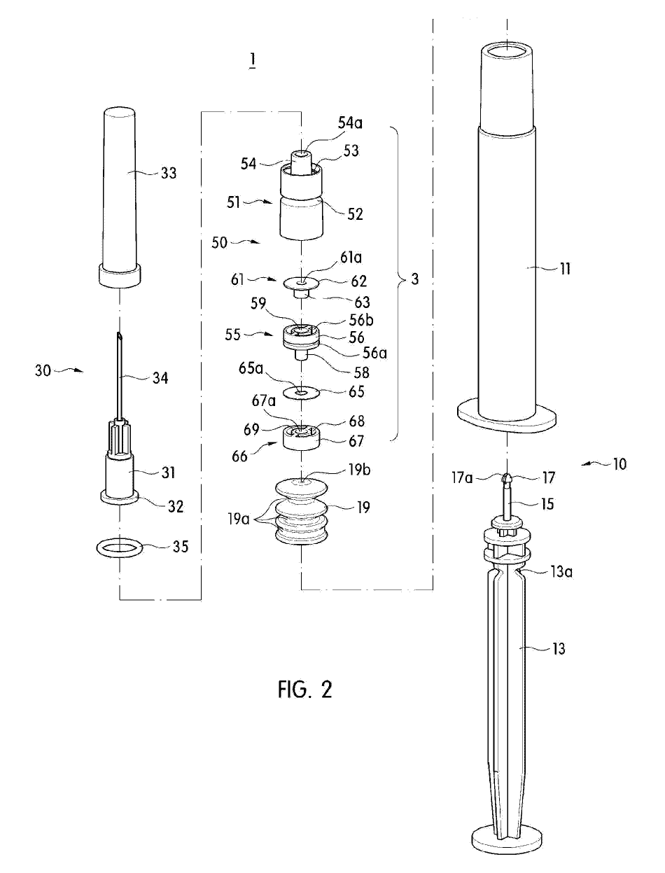

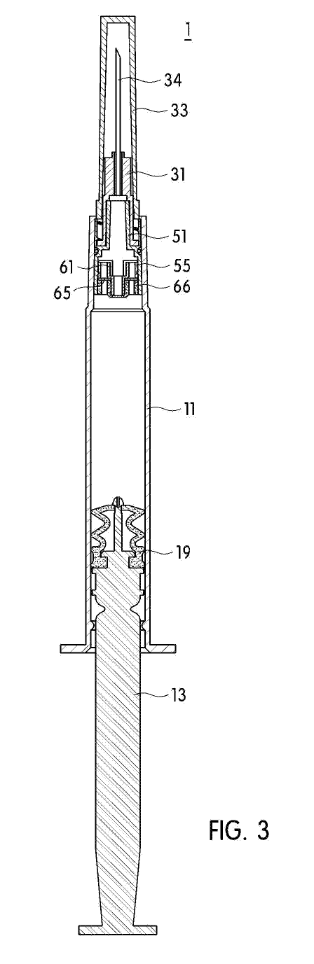

FIG. 2 is an exploded perspective view of the syringe including the filter structure for a syringe, according to one embodiment of the present invention. FIG. 3 is a cross-sectional view of the syringe including the filter structure for a syringe, according to one embodiment of the present invention.

Referring to FIGS. 1 and 2, in one embodiment of the present invention, the injection needle hub 30 may include a fixing member 31 on which an injection needle 34 is mounted and a cover 33 that covers the injection needle.

As shown in FIG. 2, in one embodiment of the present invention, the injection needle 34 may be mounted in front of the fixing member 31. Also, a first corresponding coupling portion 32 may be formed on an outer circumferential surface of a rear part of the fixing member 31.

Meanwhile, the first corresponding coupling portion 32 may be formed in a male-spiral shape and may be coupled with a first coupling portion 53 of a filter holder 51 in such a way that at least part of the fixing member 31 may be installed in the filter holder.

Here, the first coupling portion 53 may be formed in a female-spiral shape on an inner circumferential surface of a front part of the filter holder 51. However, the first coupling portion 53 and the first corresponding coupling portion 32 are not limited thereto when being screw-couplable.

Meanwhile, referring to FIGS. 2 and 3, in one embodiment of the present invention, the cylinder 11 may have a cylindrical shape with a hollow to store an injectable solution therein. Here, the filter holder 51 may be installed in a front part of the cylinder 11.

Referring to FIGS. 3, 4, 8, and 9, the filter structure 3 according to one embodiment of the present invention may include a filter portion 50 in which a first fluid path installed in the cylinder 11 and through which an injectable solution is injected is formed not to overlap with a second fluid path through which the injectable solution is discharged from the cylinder.

Meanwhile, the second fluid path may not be formed on an outer circumference of the first fluid path. Here, the first fluid path includes a through hole 54a, a second suction hole 61a, a first suction hole 59, and a third suction hole 67a and the second fluid path includes a second discharge hole 62, a first discharge hole 68, and a through hole 54a.

Through this, the filter structure 3 according to one embodiment of the present invention may simplify an injection process and may prevent glass particles of an ample from being injected into a patient by allowing an injectable solution not to pass through a filter member 65 when being injected into a syringe and to pass through the filter member when being injected into the patient.

In more detail, in one embodiment of the present invention, the filter portion 50 may be coupled with the front part of the cylinder 11 and may include the filter holder 51, a first check valve 61, a filter body 55, the filter member 65, and a second check valve 66.

Here, the filter structure 3 according to one embodiment of the present invention may include the filter portion 50 to filter out foreign substances included in the injectable solution while discharging the injectable solution.

Referring to FIG. 2, in one embodiment of the present invention, in front of the filter holder 51, the fixing member 31 on which the injection needle 34 is mounted is fixed in the cylinder 11 to allow an injectable solution to flow into the cylinder 11 through the injection needle 34 or to discharge outward from the cylinder.

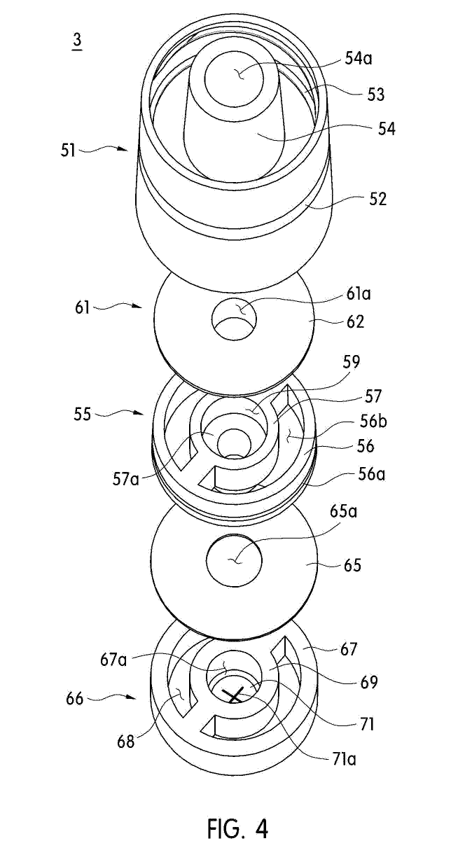

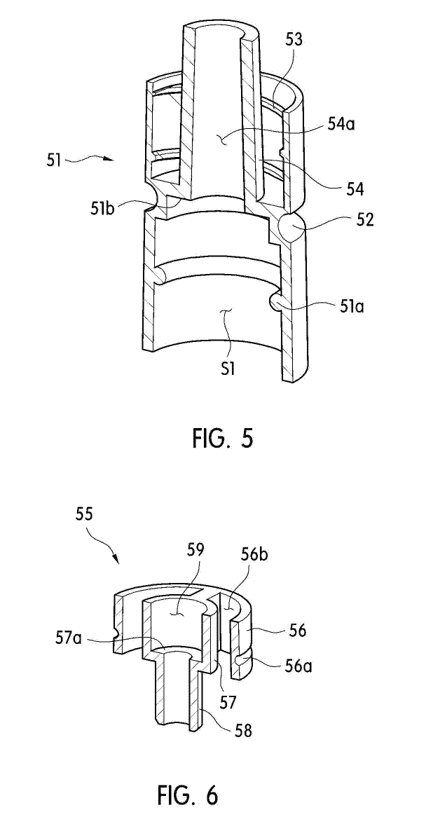

Referring to FIGS. 4 and 5, the filter holder 51 has a cylindrical shape and may include an accommodation portion S1 in which the filter portion 50 may be accommodated, at a rear part thereof.

Also, a through column portion 54 through which an injectable solution is suctioned or discharges through the injection needle 34 may be formed in a center of the filter holder 51.

Here, the through column portion 54 may have a cylinder shape that protrudes forward from the center of the filter holder 51 and may include the through hole 54a formed in a center of the through column portion to allow an injectable solution to pass therethrough.

Also, the fixing member 31 is inserted into and connected to the through column portion 54 to allow an injectable solution to flow through the through hole 54a and the injection needle 34. Here, through hole 54a may be connected to the first fluid path and the second fluid path.

Referring to FIG. 5, in one embodiment of the present invention, the filter holder 51 may have a diameter that gradually increases toward a rear not to be out of the front of the cylinder 11.

Also, an O-ring groove 52 depressed inward to seal a gap between an inside of the cylinder 11 and an outer circumferential surface of the filter holder 51 may be formed in the outer circumferential surface of the filter holder 51.

Here, an O-ring 35 is installed in the O-ring groove 52 to allow the filter holder 51 to come into close contact with the inside of the cylinder 11 to be coupled therewith.

Meanwhile, in one embodiment of the present invention, a plurality of protrusions 51a may be formed on an inner circumferential surface of the filter holder 51 to protrude inward. The plurality of protrusions 51a may be inserted into and coupled with a plurality of insertion grooves 56a formed on an outer circumferential surface of the filter body 55.

Here, the plurality of protrusions 51a may be formed corresponding to the plurality of insertion grooves 56a. Through this, the filter body 55 may be coupled with the accommodation portion S1 of the filter holder 51 to fix the filter body 55 regardless of a flow of the injectable solution.

Referring to FIG. 5, the filter holder 51 may include a holding lip 51b formed in front of the accommodation portion S1 to protrude inward.

Through this, the filter portion 50 may not deviate forward from the accommodation portion S1 of the filter holder 51. That is, the first check valve 61 accommodated in the accommodation portion S1 of the filter holder 51 may be prevented from deviating forward from the filter holder due to the holding lip 51b.

Meanwhile, referring to FIGS. 2 and 5, the first check valve 61, the filter body 55, the filter member 65, and the second check valve 66 may be accommodated in the accommodation portion S1 formed in the rear of the filter holder 51.

Referring to FIGS. 2 and 3, in one embodiment of the present invention, the fixing member 31 may be coupled with a front part based on the holding lip 51b of the filter holder 51 and the first check valve 61, the filter body 55, the filter member 65, and the second check valve 66 may be sequentially coupled with a rear part but are not limited to this order.

The filter structure 3 according to one embodiment of the present invention includes the first check valve 61 and the second check valve 66 to allow the first fluid path and the second fluid path to open only in different directions due to a check valve structure.

Meanwhile, in one embodiment of the present invention, the first check valve 61 may allow an injectable solution to flow into the cylinder 11 through the first fluid path by obstructing the second fluid path. Also, the second check valve 66 may be connected to the first check valve 61 and may discharge an injectable solution outward from the cylinder 11 through the second fluid path by obstructing the first fluid path.

Here, the filter member 65 may be installed at the second fluid path and may filter the injectable solution that passes through and discharges from the second check valve 66.

Referring to FIGS. 4 and 6, the filter structure 3 according to one embodiment of the present invention may include the filter body 55.

Referring to FIG. 6, the filter body 55 has a longitudinal cross section in a T shape and is positioned between the first check valve 61 and the filter member 65. Also, the filter body 55 may include a first outer barrel portion 56, a first inner barrel portion 57, and an extending pipe 58.

Also, a front part of the filter body 55 supports the first check valve 61 and a rear part thereof allows the filter member to come into close contact with the second check valve 66.

Referring to FIGS. 4 and 6, the first outer barrel portion 56 has a barrel shape and includes the plurality of insertion grooves 56a formed on an outer circumferential surface of the first outer barrel portion to be inserted into and coupled with the accommodation portion S1 of the filter holder 51. The plurality of insertion grooves 56a are formed corresponding to the plurality of protrusions 51a formed on the inner circumferential surface of the filter holder 51 and to be insertion-coupled therewith.

Meanwhile, the first inner barrel portion 57 connected to the first outer barrel portion 56 may be formed in the first outer barrel portion 56. The first inner barrel portion 57 has a barrel shape, and a first discharge hole 56b may be formed at the second fluid path between the first outer barrel portion 56 and the first inner barrel portion 57.

Referring to FIG. 6, the first inner barrel portion 57 is connected to the extending pipe 58 at a rear thereof. The extending pipe 58 may be formed to extend behind the first inner barrel portion 57. Here, the first suction hole 59 may be formed at the first fluid path in centers of the first inner barrel portion 57 and the extending pipe 58.

Meanwhile, in one embodiment of the present invention, the filter body 55 may include a pair of such first discharge holes 56b around the first suction hole 59 formed in the center to longitudinally pass through the filter body.

Here, referring to FIG. 3, the first through holes 56b may be formed around the first suction hole 59 to extend in a circumferential direction to surround at least part of the first suction hole 59.

Meanwhile, in one embodiment of the present invention, a supporting lip 57a is formed on an inner circumferential surface of the first inner barrel portion 57 to extend inward. Here, when a coupling shaft member 15 is inserted into the first suction hole 59, a holding protrusion 17 is supported by the supporting lip in such a way that the coupling shaft member and the filter body 55 may be coupled with each other.

Meanwhile, the first check valve 61 is positioned in front of the filter body 55, that is, the first outer barrel portion 56 and the first inner barrel portion 57. Here, the first check valve 61 is inserted into the first suction hole 59 and connected to the filter body 55. Through this, the filter body 55 may support a discus portion 62 of the first check valve 61.

Referring to FIG. 4, in one embodiment of the present invention, the first check valve 61 has a longitudinal cross section in a T shape and may include the discus portion 62 and a first column portion 63.

In one embodiment of the present invention, the discus portion 62 has a discus shape and may be disposed to come into close contact with a front surface of the filter body 55. Also, the discus portion 62 is formed at a front part of the check valve 61 and may selectively obstruct the first discharge hole 56b of the filter body 55 when an injectable solution flows inward or discharges.

Meanwhile, the discus portion 62 is disposed to be spaced behind from the holding lip 51b of the filter holder 51 to form a mobile space S2 between the holding lip and the discus portion to space the first check valve 61 in close contact with the first discharge hole 56b from the first discharge hole 56b.

That is, when an injectable solution discharges from the cylinder 11, the discus portion 62 of the first check valve 61 moves toward the holding lip 51b of the filter holder 51, the mobile space S2 is formed between the first check valve and the filter body 55, and the injectable solution may discharge through the mobile space S2.

Meanwhile, in one embodiment of the present invention, the discus portion 62 of the first check valve 61 is formed of an elastic material to selectively obstruct the first discharge hole 56b of the filter body 55 by an elastic deformation of the discus portion 62.

In one embodiment of the present invention, the first column portion 63 has a cylindrical shape and may be formed to protrude backward from the discus portion 62. Also, the first column portion 63 may be inserted into the first suction hole 59 of the filter body 55 and may be coupled with the filter body.

Meanwhile, the second suction hole 61a may be formed in a center of the first check valve 61, that is, at the discus portion 62 and the first column portion 63 to suction an injectable solution.

Here, when the injectable solution is suctioned in the cylinder 11, the discus portion 62 of the first check valve 61 obstructs the first discharge hole 56b of the filter body 55 in such a way that the injectable solution may flow into the cylinder 11 through the first suction hole 59 and the second suction hole 61a.

Referring to FIGS. 3 and 4, the first column portion 63 of the first check valve 61 is inserted into the first suction hole 59 of the filter body 55 in such a way that the first suction hole may be connected to the second suction hole 61a.

Meanwhile, in one embodiment of the present invention, the filter member 65 has a discus shape and an opening 65a in which an injectable solution is suctioned may be formed in a center thereof. Also, the extending pipe 58 of the filter body 55 is inserted into the opening 65a of the filter member 65 in such a way that the filter member may be fixed between the filter body and the second check valve 66.

Meanwhile, the filter member 65 may be formed to cover a second discharge hole 68 of the second check valve 66 to allow an injectable solution to pass through the filter member while passing through the second discharge hole 68.

That is, the filter structure 3 according to one embodiment of the present invention may easily suction an injectable solution during suction by allowing the injectable solution to pass through the filter member 65 only when discharging.

Also, the filter member 65 may be formed of various materials regardless of type when having density capable of filtering out foreign substances included in an injectable solution that passes through the second discharge hole 68.

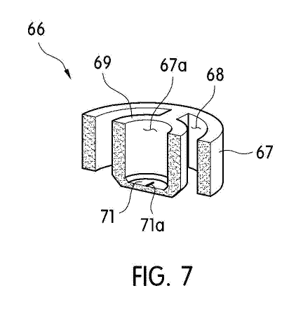

Referring to FIG. 7, in one embodiment of the present invention, the second check valve 66 has a cylindrical shape and may include a second outer barrel portion 67 and a second inner barrel portion 69.

Meanwhile, the second outer barrel portion 67 has a barrel shape and may be accommodated in the accommodation portion S1 of the filter holder 51. The second inner barrel portion connected to the second outer barrel portion may be formed in the second outer barrel portion 67.

Also, the second discharge hole 68 may be formed at the second fluid path between the second outer barrel portion 67 and the second inner barrel portion 69 to discharge an injectable solution.

Meanwhile, referring to FIG. 7, the third suction hole 67a may be formed at the first fluid path in a center of the second inner barrel portion 67 to suction an injectable solution.

Here, the extending pipe 58 of the filter body 55 may be inserted into the third suction hole 67a to allow the filter member 65 to come into close contact with a front surface of the second check valve 66. Also, through this, the first suction hole 59 and the third suction hole 67a may be connected to each other.

Referring to FIG. 4, a pair of such second discharge holes 68 may be formed around the third suction hole 67a formed in the second check valve 66 in a longitudinal direction of the second check valve.

Referring to FIG. 4, the second discharge holes 68 may be formed to extend in a circumferential direction and may be formed around the third suction hole 67a to surround at least part of the third suction hole.

Meanwhile, the filter structure 3 according to one embodiment of the present invention may include a section 69a at least partially cut and including a cutting line 71a on one side, that is, a rear surface of the third suction hole 67a.

Through this, the filter structure 3 according to one embodiment of the present invention may allow an injectable solution to selectively pass through the third suction hole 67a and pass the filter member 65 only when discharging.

Referring to FIG. 3, in one embodiment of the present invention, the cutting line 69a may be formed by making a cut in a linear or cross shape inward from the outside to be well opened during suction and tightly closed during discharge.

Here, in one embodiment of the present invention, the section 71 may be formed of an elastically deformable material.

Meanwhile, in one embodiment of the present invention, the first check valve 61 and the second check valve 66 may be formed of an elastically deformable material for opening and closing a flow path, for example, a silicone material. Accordingly, it is necessary to form the first check valve 61 and the second check valve 66 using a material having excellent elasticity and no harms on hygiene.

Meanwhile, referring to FIG. 2, in the case of the filter structure 3 according to one embodiment of the present invention, the cylinder 11 may be installed inside. That is, the filter holder 51 is coupled with the front part of the cylinder 11 and the piston 10 is inserted into and coupled with the rear part of the cylinder 11 to be vertically movable.

In one embodiment of the present invention, the piston 10 may include a piston body 13 and a gasket 19.

Referring to FIGS. 2 and 3, in one embodiment of the present invention, the piston body 13 may include the coupling shaft member 15 that protrudes forward to allow the gasket 19 to be coupled with a front part thereof.

Meanwhile, referring to FIG. 2, the coupling shaft member 15 has a cylindrical shape and is formed in a center of the piston body 13 to be insertion-coupled with the gasket 19.

That is, the coupling shaft member 15 may include the holding protrusion 17 formed at a front end thereof to protrude forward to be supported by the supporting lip 57a formed on the inner circumferential surface of the first inner barrel portion 57.

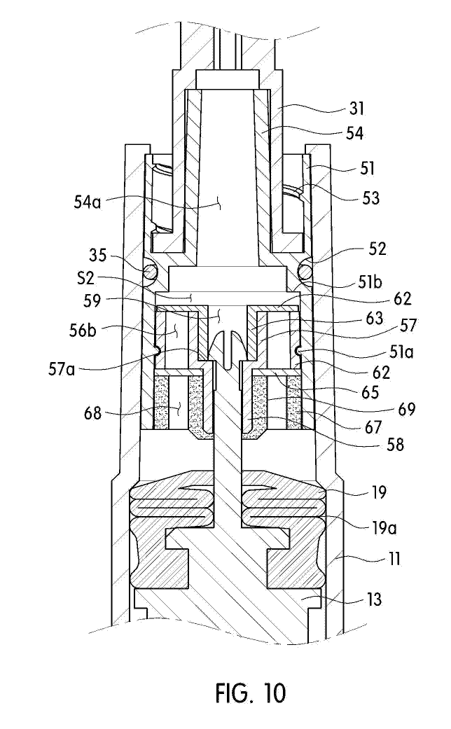

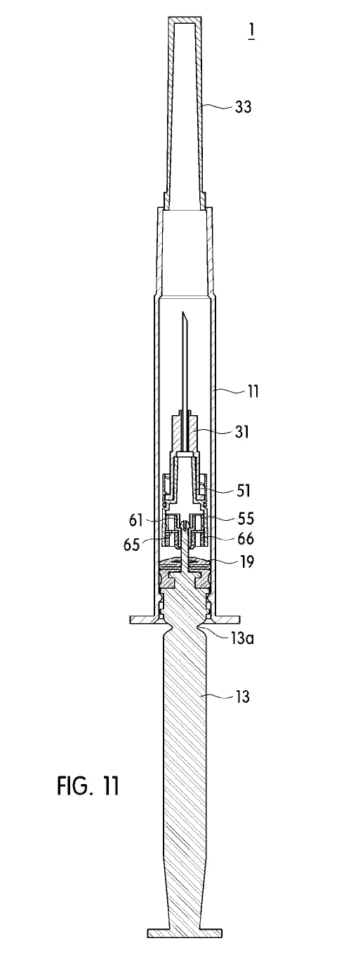

Meanwhile, referring to FIGS. 10 and 11, when the coupling shaft member 15 passes through the third suction hole 67a, a rear section of the holding protrusion 17 may be supported by the supporting lip 57a.

Also, the holding protrusion 17 may include a coupling groove 17a at a front part. The coupling groove 17a may be formed to be depressed backward in such a way that the holding protrusion 17 may be formed in the shape of tongs.

Here, the holding protrusion 17 may be elastically deformed and increase a supporting force of being supported by the supporting lip 57a.

Referring to FIG. 11, in one embodiment of the present invention, the piston body 13 may include a bent groove 13a formed to be spaced backward apart from the coupling shaft member 15.

Here, a pair of such bent grooves 13a may be formed to be depressed toward an inside of the piston body 13 to be easily bent when an external force is applied to the piston body in a perpendicular direction.

Referring to FIG. 2, in one embodiment of the present invention, the gasket 19 may be inserted in the cylinder 11 to be vertically movable and may be manufactured using rubber having an excellent sealing property or a flexible synthetic resin. Here, the gasket 19 moves while being inserted to come into close contact with an inner circumferential surface of the cylinder 11.

Meanwhile, in one embodiment of the present invention, the gasket 19 has a cylindrical shape and may include a wrinkle portion 19a formed inward on an outer circumferential surface of the gasket. Here, the wrinkle portion 19a may be formed in an accordion shape.

The piston body 13 according to one embodiment of the present invention may be inserted into the cylinder 11 through the wrinkle portion 19a of the gasket 19 to come into close contact therewith to prevent a leakage of an injectable solution.

Meanwhile, referring to FIG. 2, in one embodiment of the present invention, a coupling hole 19b may be formed in a center of the gasket 19 to allow the coupling shaft member 15 of the filter body 55 to be inserted and coupled. Here, when the coupling shaft member 15 is inserted into the coupling hole 19b of the gasket 19, the holding protrusion 17 may be held and supported by a front surface of the gasket.

Hereinafter, an operation state of a syringe including the filter structure according to one embodiment of the present invention will be described.

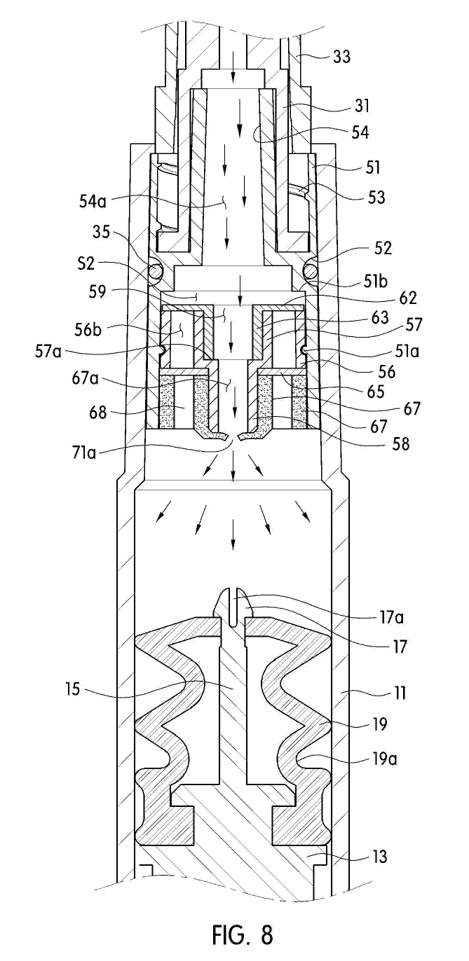

Referring to FIG. 8, in one embodiment of the present invention, when the piston 10 is pulled backward, a relatively low pressure is formed in a space between the second check valve 66 and the gasket 19 of the piston 10 in such a way that the discus portion 62 of the first check valve 61 comes into close contact with the first discharge hole 56b of the filter body 55.

Here, when an injectable solution is suctioned in, since a pressure increases in such a way that the cutting line 71a is opened and the injectable solution is suctioned in the cylinder through the third suction hole 67a.

In this state, the injectable solution passes through the first fluid path formed in the center, for example, the through hole 54a, the second suction hole 61a, the first suction hole 59, and the third suction hole 67a and flows into the space between the second check valve 66 and the gasket 19 of the piston 10.

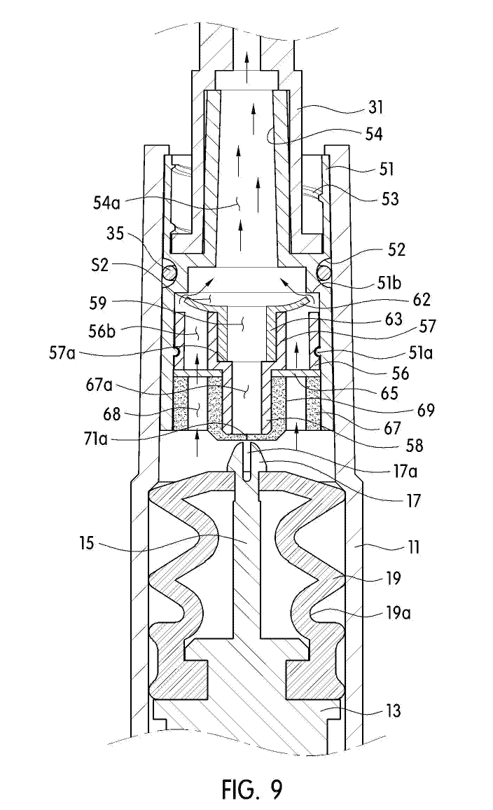

Also, referring to FIG. 9, in one embodiment of the present invention, when the piston 10 is pushed forward, a relatively high pressure is formed in the space between the second check valve 66 and the gasket 19 of the piston 10 in such a way that the discus portion 62 of the first check valve 61 is spaced apart from the first discharge hole 56b of the filter body 55.

Here, when an injectable solution discharges, the pressure decreases in such a way that the cutting line 71a is obstructed and the injectable solution discharges through the second discharge hole 68 and the filter member 65.

In this state, the injectable solution passes through the second fluid path formed at an outer circumferential portion of the first fluid path, for example, the second discharge hole 62, the first discharge hole 68, and the through hole 54a and discharges outward from the cylinder.

Meanwhile, referring to FIGS. 7 and 8, the syringe 1 that discharges all of an injectable solution further pushes the piston 10 forward to allow the holding protrusion 17 of the coupling shaft member 15 to be held by the supporting lip 57a of the filter body 55.

After that, the piston 10 is pulled backward and the injection needle 34 is completely inserted into the cylinder 11 to expose the bent groove 13a formed at the piston body 13 outward from the cylinder.

Here, when the piston body 13 is cut by applying an external force to the bent groove 13a, since the piston 10 can not be pulled any more, it is impossible to take out the injection needle 34 from the cylinder 11.

Through this, since the syringe 1 including the filter structure according to one embodiment of the present invention prevents an injection needle from being reused and simultaneously deviating outward, a negligent accident of being stuck with the injection needle may be prevented during a process of discarding the syringe.

According to one embodiment of the present invention, a syringe including a filter structure for a syringe may simplify an injection process and prevent foreign substances from being injected into a patient by allowing an injectable solution not to pass through a filter member when being injected into the syringe and to pass through the filter member when being injected into the patient.

Although one embodiment of the present invention has been described above, the concept of the present invention is not limited to the embodiment disclosed in the specification and other embodiments may be easily provided by one of ordinary skill in the art through additions, changes, deletions, supplements, and the like but they will be included within the conceptual scope of the present invention.

### INDUSTRIAL APPLICABILITY

A syringe including a filter structure for a syringe, according to one embodiment of the present invention, may simplify an injection process and prevent foreign substances from being injected into a patient by allowing an injectable solution not to pass through a filter member when being injected into the syringe and to pass through the filter member when being injected into the patient.

Since the syringe including the filter structure according to one embodiment of the present invention prevents an injection needle from being reused and simultaneously deviating outward, a negligent accident of being stuck with the injection needle may be prevented during a process of discarding the syringe.

We claim:

1. A filter structure for a syringe for filtering an injectable solution that flows backward from an injection needle coupled with a front of a cylinder, the filter structure comprising a filter portion, a second fluid path formed in the filter portion on an outer circumferential portion of a first fluid path to prevent the first fluid path from overlapping with the second fluid path, wherein the injectable solution discharges from the cylinder through the second fluid path, the first fluid path installed in the cylinder, the first fluid path is connected to the injection needle, and the injectable solution flows through the first fluid path,
wherein the filter portion comprises:
a first check valve, wherein the first check valve is connected to the injection needle and allows the injectable solution to flow into the cylinder through the first fluid path by obstructing the second fluid path;
a second check valve, wherein the second check valve obstructs the first fluid path to discharge the injectable solution outward from the cylinder through the second fluid path;
a filter member, wherein the filter member is installed at the second fluid path and filters the injectable solution passing through the second check valve and discharging; and
a filter body, wherein the filter body is positioned between the first check valve and the filter member and has a front part coupled with the first check valve and a rear part coupled with the second check valve to allow the filter member to come into close contact with the second check valve.

2. The filter structure of claim 1, comprising a filter holder installed in the cylinder and has a front part coupled with the injection needle and a rear part with an accommodation portion to accommodate the filter portion,
wherein the filter holder has a cylindrical shape with a through hole formed at a center, wherein the injectable solution passes through the through hole, and the through hole is connected to the first fluid path and the second fluid path.

3. The filter structure of claim 2, wherein a holding lip formed to protrude toward an inside of the filter holder is provided at a front part of the accommodation portion to prevent the filter portion accommodated in the accommodation portion from deviating forward.

4. The filter structure of claim 3, wherein an O-ring groove formed to be depressed inward for sealing a gap between an inside of the cylinder and an outer circumferential surface of the filter holder is provided on the outer circumferential surface of the filter holder.

5. The filter structure of claim 3, wherein the filter body comprises:
a first outer barrel portion having a barrel shape;
a first inner barrel portion connected to the first outer barrel portion and formed in the first outer barrel portion; and
an extending pipe formed to extend backward from the first inner barrel portion.

6. The filter structure of claim 5, wherein the filter body comprises a first suction hole formed at the first fluid path in centers of the first inner barrel portion and the extending pipe and a first discharge hole formed at the second fluid path between the first outer barrel portion and the first inner barrel portion.

7. The filter structure of claim 5, wherein a plurality of protrusions are formed on an inner circumferential surface of the filter holder, and
wherein a plurality of insertion-grooves formed corresponding to the plurality of protrusions are formed on an outer circumferential surface of the first outer barrel portion in such a way that the filter body is coupled with the accommodation portion of the filter holder.

8. The filter structure of claim 6, wherein the first check valve comprises:
a discus portion, wherein the discus portion has a discus shape and is disposed to come into close contact with a front surface of the filter body to selectively obstruct the first discharge hole when the injectable solution flows in or discharges; and
a first column portion formed to protrude backward from the discus portion and inserted into and coupled with the first suction hole, and
wherein a second suction hole is formed at the first fluid path in centers of the discus portion and the first column portion.

9. The filter structure of claim 8, wherein the discus portion is disposed to be spaced backward apart from the holding lip in such a way that a mobile space is formed between the holding lip and the discus portion to allow the discus portion to be spaced apart from the first discharge hole.

10. The filter structure of claim 6, wherein the second check valve comprises:
a second outer barrel portion having a barrel shape; and
a second inner barrel portion connected to the second outer barrel portion and formed in the second outer barrel portion,
wherein a third suction hole is formed at the first fluid path in a center of the second inner barrel portion, the extending pipe is inserted into the third suction hole and the third suction hole allows the filter member to come into close contact with a front surface of the second check valve, and
wherein a second discharge hole is formed at the second fluid path between the second outer barrel portion and the second inner barrel portion.

11. The filter structure of claim 10, comprising a section including a cutting line and at least partially cut at one side of the third suction hole,
wherein the section selectively obstructs the third suction hole when the injectable solution flows in or discharges.

12. The filter structure of claim 10, wherein the first discharge hole and the second discharge hole are formed to extend in a circumferential direction and to surround at least parts of the first suction hole and the third suction hole.

13. A syringe comprising:
a fixing member having a front, wherein an injection needle is mounted on the front;
a cylinder, wherein the fixing member is detachably coupled with the cylinder;
a filter structure installed in the cylinder and coupled with the fixing member; and
a piston, wherein the piston moves forward or backward while being at least partially inserted in the cylinder to allow an injectable solution to flow into the cylinder or to discharge outward from the cylinder,
wherein the filter structure comprises a filter portion, a first fluid path is installed in the cylinder, the injectable solution flows through the first fluid path, a second fluid path is formed in the filter portion on an outer circumferential portion of a first fluid path to prevent the first fluid path from overlapping with the second fluid path, the injectable solution discharges from the cylinder through the second fluid path, the first fluid path is connected to the injection needle,
wherein the filter portion comprises:
a first check valve, wherein the first check valve is connected to the injection needle and allows the injectable solution to flow into the cylinder through the first fluid path by obstructing the second fluid path;
a second check valve, wherein the second check valve obstructs the first fluid path to discharge the injectable solution outward from the cylinder through the second fluid path;
a filter member, wherein the filter member is installed at the second fluid path and filters the injectable solution passing through the second check valve and discharging; and
a filter body, wherein the filter body is positioned between the first check valve and the filter member and has a front part coupled with the first check valve and a rear part coupled with the second check valve to allow the filter member to come into close contact with the second check valve.

14. The syringe of claim 13, wherein a screw thread is formed on an inner circumferential surface of a front of the filter holder and screw-coupled with a rear part of the fixing member in such a way that at least part of the fixing member is installed in the filter holder.

15. The syringe of claim 13, wherein the piston comprises:
a piston body inserted into the cylinder and moves in one direction;
a gasket coupled with a front end of the piston body and inserted into the cylinder to come into close contact with the cylinder; and
a coupling shaft member positioned at a front end portion of the piston body, formed to protrude forward, and coupling the gasket with the piston body.

16. The syringe of claim 15, wherein a holding protrusion with a coupling groove formed to be depressed backward is formed at an end of the coupling shaft member,
wherein a supporting lip formed to extend inward is provided on an inner circumferential surface of the first inner barrel portion, and
wherein a rear end surface of the holding protrusion is supported by the supporting lip when the coupling shaft member passes through the third suction hole.

17. The syringe of claim 16, wherein the holding protrusion is formed of an elastically deformable material.

18. The syringe of claim 15, wherein the piston body comprises a bent groove formed to be depressed inward between the coupling shaft member and a rear end of the piston body.
